(19) United States
(12) Patent Application Publication
(10) Pub. No.: US 2024/0173259 A1
(43) Pub. Date: May 30, 2024

(54) ORAL FORMULATION COMPRISING 1-(3-CYANO-1-ISOPROPYL-INDOLE-5-YL)PYRAZOLE-4-CARBOXYLIC ACID AND METHOD FOR PREPARING SAME

(71) Applicant: LG CHEM, LTD., Seoul (KR)

(72) Inventors: Seok Cheol YOO, Daejeon (KR); Joomyung JANG, Daejeon (KR); Ree Sun KIM, Daejeon (KR); Jin A SEO, Daejeon (KR)

(73) Assignee: LG CHEM, LTD., Seoul (KR)

(21) Appl. No.: 18/255,241

(22) PCT Filed: Nov. 30, 2021
(86) PCT No.: PCT/KR2021/017843

§ 371 Date: May 31, 2023

(30) Foreign Application Priority Data
Dec. 1, 2020 (KR) 10-2020-0165790

(51) Int. Cl. A61K 9/20; A61K 31/4155

(52) U.S. Cl. CPC A61K 9/2009; A61K 31/4155

(57) ABSTRACT

The present invention relates to an oral formulation comprising an API selected from 1-(3-cyano-1-isopropyl-indol-5-yl)pyrazol-4-carboxylic acid or a pharmaceutically acceptable salt thereof in high content.

Since the oral formulation according to the present invention has a high content and excellent physical properties by comprising a glidant in excipients, economic efficiency and the convenience of administration can be increased.

### TECHNICAL FIELD

The present application claims the benefit of priority based on Korean Patent Application No. 10-2020-0165790 filed on Dec. 1, 2020, the entire disclosure of which are incorporated herein by reference its entirely.

The present invention relates to an oral formulation with excellent physical properties, which comprising an active pharmaceutical ingredient (API) selected from 1-(3-cyano-1-isopropyl-indol-5-yl)pyrazol-4-carboxylic acid or a pharmaceutically acceptable salt thereof with high content, which is useful as a xanthine oxidase inhibitor that can prevent the deposition of uric acid in the body, and to a preparation method thereof.

### BACKGROUND ART

Xanthine oxidase is an enzyme that converts hypoxanthine to xanthine, and also converts the formed xanthine to uric acid. It is known that when there is too much uric acid in the body, it causes various diseases, including gout and the like.

Therefore, substances that inhibit the activity of xanthine oxidase can effectively treat xanthine oxidase-related diseases such as hyperuricacidemia, gout, heart failure, cardiovascular diseases, high blood pressure, diabetes, kidney diseases, joint diseases, and inflammatory bowel disease.

Meanwhile, with respect to a substance that inhibits the activity of xanthine oxidase, KR 10-1751325 (Patent Document 1) provides 1-(3-cyano-1-isopropyl-indol-5-yl)pyrazol-4-carboxylic acid, and a method for preparing the compound, and KR 10-1424013 (Patent Document 2) provides various types of crystalline forms obtained by using various solvents, and a preparation method thereof.

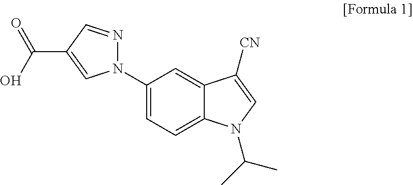 [SMILES: CC(C)n1cc(C#N)c2cc(-n3cc(C(=O)O)cn3)ccc21]

However, it has not been reported for an oral formulation comprising 1-(3-cyano-1-isopropyl-indol-5-yl)pyrazol-4-carboxylic acid or a pharmaceutically acceptable salt thereof as an active pharmaceutical ingredient (API), and in particular, there has been no report on the oral formulation comprising the API with high content.

Formulations comprising API with high content cannot generally be said to have excellent fluidity and tableting properties. Accordingly, in order to overcome these disadvantages, it is common to use wet granulation or dry granulation method or to increase the amounts of excipients to reduce the API ratio.

However, there is a problem that the high content formulation can increase the manufacturing time and production cost by performing a granulation process for improving fluidity, as well as increase the total weight and volume of the formulation by increasing the amount of excipients. So conventional high content formulation shows increasing the production cost and lowering the patient's convenience in taking. In addition, it is common knowledge in the art that even if a granulation process is added or the amounts of excipients is increased to prepare the high content formulation, the formulation assay may be affected by the imbalance between the excipients and the high content API.

Therefore, it is necessary to develop an oral formulation with excellent physical properties while comprising 1-(3-cyano-1-isopropyl-indol-5-yl)pyrazol-4-carboxylic acid or a pharmaceutically acceptable salt thereof as API with high content.

### PRIOR ART DOCUMENTS

### Patent Documents

1. KR 10-1751325 (Jun. 21, 2017), Novel compounds effective as xanthine oxidase inhibitors, method for preparing the same, and pharmaceutical composition comprising the same
2. KR 10-1424013 (Jul. 22, 2014), 1-(3-cyano-1-isopropyl-indol-5-yl)pyrazole-4-carboxylic acid crystalline form and the producing method thereof

### DISCLOSURE

### Technical Problem

It is an object of the present invention to provide an oral formulation having excellent physical properties, comprising an excess of 1-(3-cyano-1-isopropyl-indol-5-yl)pyrazol-4-carboxylic acid or a pharmaceutically acceptable salt thereof, as an API.

### Technical Solution

The present invention provides an oral formulation with excellent physical properties, comprising an API selected from 1-(3-cyano-1-isopropyl-indol-5-yl)pyrazol-4-carboxylic acid or a pharmaceutically acceptable salt thereof, and excipients, in particular a glidant in the excipients, and a method for preparing the oral formulation.

The glidant comprised in the oral formulation of the present invention is SiO₂ selected from the group consisting of colloidal silicon dioxide, hydrated silicon dioxide and combinations thereof, and the content of the glidant per formulation is 0.2 to 1% by weight, or 0.2 to 0.8% by weight, based on the total weight of the formulation.

The content of API comprised in the oral formulation of the present invention is 30 to 50% by weight based on the total weight of the formulation.

The API assay of the oral formulation of the present invention is 95% or more.

The oral formulation of the present invention is used for the treatment or prophylaxis of xanthine oxidase-related diseases selected from the group consisting of hyperuricacidemia, gout, heart failure, cardiovascular diseases, high blood pressure, diabetes, kidney diseases, inflammation, joint diseases, and inflammatory bowel disease.

### Advantageous Effects

The oral formulation comprising 1-(3-cyano-1-isopropyl-indol-5-yl)pyrazol-4-carboxylic acid or a pharmaceutically acceptable salt thereof as an API according to the present invention has excellent physical properties and maintain high formulation assay with a glidant in the excipients despite the high API content.

In addition, the oral formulation according to the present invention comprises a high content of 1-(3-cyano-1-isopropyl-indol-5-yl)pyrazol-4-carboxylic acid or a pharmaceutically acceptable salt thereof, which is an API, and thus is not only economical, but also can increase the convenience of administration.

### DESCRIPTION OF DRAWINGS

FIG. 1 shows API assay per unit tablet depending on glidant (SiO₂) content.

### BEST MODE

Hereinafter, the present invention will be described in more detail.

All technical terms used in the present invention, unless otherwise defined, are used in the same meaning as commonly understood by those of ordinary skill in the related field of the present invention. In addition, although preferred methods and samples are described herein, similar or equivalent ones are also included in the scope of the present invention. The disclosure of all publications incorporated herein by reference are hereby incorporated by reference in their entirety.

The inventors of the present invention have continued research in various ways in order to increase the content of the API and maintain or increase the physical properties in the oral formulation comprising 1-(3-cyano-1-isopropyl-indol-5-yl)pyrazol-4-carboxylic acid or a pharmaceutically acceptable salt thereof as API. And as a result, the inventors of the present invention have developed an oral formulation with high API assay while excellent physical properties comprising a glidant in excipients.

In this case, even if it comprises an excess of API compared to the oral formulation that does not comprise the glidant, since the physical properties are maintained when formulated as an uncoated tablet, there is no need to increase the content of excipients according to the increase of API content, and thus it has the advantage of being economical as well as being able to make a high-content oral formulation with increased convenience of administration without increasing the size of the formulation. In addition, there is an advantage that since it has a high API assay, the quality of the final product is also excellent.

Therefore, the present invention provides an oral formulation comprising a high content of the API selected from 1-(3-cyano-1-isopropyl-indol-5-yl)pyrazol-4-carboxylic acid or a pharmaceutically acceptable salt thereof, with a glidant in excipients.

As used herein, “pharmaceutically acceptable salt” refers to a salt form of a compound that does not cause serious irritation to the organism to which the compound is administered and does not impair the biological activities and physical properties of the compound. 1-(3-cyano-1-isopropyl-indol-5-yl)pyrazol-4-carboxylic acid, which is the API comprised in the oral formulation of the present invention, can be converted to its salt by conventional methods.

As used herein, the term “API assay” or “assay” refers to the amount of an active ingredient in a formulation or tablet. The assay is an important quality characteristic of the final product.

In oral formulations such as tablets or granular capsules, it is common to minimize the size per unit dosage form for easy swallowing even if a high content API is included. However, it is the technical common knowledge of those of ordinary skill in the art that when the API is comprised in high content, not only physical properties such as fluidity and tableting properties are generally impaired, but also the assay of the formulation may be affected by the imbalance between the high content API and each excipient.

In fact, in an oral formulation comprising 1-(3-cyano-1-isopropyl-indol-5-yl)pyrazol-4-carboxylic acid as an API, the acceptable content of the API in the oral formulation (especially tablets) with pharmaceutically acceptable physical properties is only around 30%, and when more than 30% of the content is included, there was a problem that physical properties such as tableting properties are rapidly deteriorated.

In the present invention, various studies were conducted by combining various excipients for oral formulations with excellent or good physical stability while comprising an API with high content, selected from 1-(3-cyano-1-isopropyl-indol-5-yl)pyrazol-4-carboxylic acid or a pharmaceutically acceptable salt there.

As a result, in the case of including a glidant as an excipient, an oral formulation with high assay as well as excellent physical stability was developed despite including a high content of API.

Accordingly, one aspect of the present invention provides an oral formulation comprising i) 1-(3-cyano-1-isopropyl-indol-5-yl)pyrazol-4-carboxylic acid or a pharmaceutically acceptable salt thereof as an API, and ii) a glidant in excipients.

In the present invention, the glidant may be comprised in an amount of greater than 0% by weight and less than 1% by weight, 0.1% by weight or more and less than 1% by weight, 0.2% by weight or more and less than 1% by weight, greater than 0% by weight and 0.9% by weight or less, 0.1% by weight or more and 0.9% by weight or less, 0.2% by weight or more and 0.9% by weight or less, greater than 0% by weight and 0.8% by weight or less, 0.1% by weight or more and 0.8% by weight or less, or 0.2% by weight or more and 0.8% by weight or less, by weight based on the total weight of the formulation. In addition, the glidant may be comprised in an amount of 0.1% by weight, 0.2% by weight, 0.3% by weight, 0.4% by weight, 0.5% by weight, 0.6% by weight, 0.7% by weight, 0.8% by weight, or 0.9% by weight based on the total weight of the formulation.

In the present invention, the glidant may be selected from the group consisting of colloidal silicon dioxide, hydrated silicon dioxide, talc and combinations thereof, but is not limited thereto. However, when talc is used as a glidant, since hardness tends to decrease and friability tends to increase when formulated as tablets, it is preferable to use a SiO₂ series such as colloidal silicon dioxide, hydrated silicon dioxide, or a combination thereof as the glidant.

In the present invention, the oral formulation further comprises one or more excipients selected from a pharmaceutically acceptable diluent, disintegrant, binder, stabilizer, lubricant and the like as an excipient.

The diluent, disintegrant, binder, stabilizer, lubricant and the like may be any ones known to be commonly used in the art. The diluent may be used in an amount of 30 to 50% by weight, 40 to 50% by weight, or 45 to 50% by weight, based on the total weight of the oral formulation. The disintegrant may be used in an amount ranging from 1 to 30% by weight, 1 to 20% by weight, 1 to 10% % by weight or 1 to 5% by weight, based on the total weight of the oral formulation. The binder may be used in an amount ranging from 1 to 30% by weight, 1 to 20% by weight, 1 to 10% % by weight, or 1 to 5% by weight, based on the total weight of the oral formulation. The stabilizer may be used in an amount ranging from 0.1 to 10% by weight, 0.3 to 5% by weight, or 0.5 to 4% by weight, based on the total weight of the oral formulation. The lubricant may be used in an amount ranging from 0.1 to 10% by weight, 0.3 to 5% by weight, or 0.5 to 4% by weight, based on the total weight of the oral formulation.

For example, the diluent may be selected from the group consisting of microcrystalline cellulose (MCC), lactose monohydrate, lactose anhydrous, lactose, starch, mannitol, carboxymethyl cellulose, sorbitol, and combinations thereof, but is not limited thereto. The disintegrant may be selected from the group consisting of low-substituted hydroxypropyl cellulose, crospovidone, croscarmellose sodium, sodium starch glycolate, F-Melt®, and combinations thereof, but is not limited thereto. The binder may be selected from the group consisting of hydroxypropyl cellulose, hydroxypropyl methylcellulose, hypromellose, polyvinyl acetic acid, povidone, polyvinylpyrrolidone, copovidone, Macrogol, sodium lauryl sulfate, light anhydrous silicic acid, synthetic aluminum silicate, calcium silicate or silicate derivatives such as magnesium metasilicate aluminate, phosphate such as calcium hydrogen phosphate, carbonate such as calcium carbonate, pregelatinized starch, gums such as acacia gum, gelatin, cellulose derivatives such as ethyl cellulose, and mixtures thereof, but is not limited thereto. The stabilizer may be selected from the group consisting of BHT, BHA, ascorbic acid, tocopherol, EDTA, and mixtures thereof, but is not limited thereto. The lubricant may be selected from the group consisting of magnesium stearate, silicon dioxide, talc, light anhydrous silicic acid, sodium stearyl fumarate, and combinations thereof, but is not limited thereto.

The oral formulation can be administered once a day and can be taken daily.

The content of API comprised in the oral formulation is 30 to 50% by weight, 35 to 50% by weight, 40 to 50% by weight, 45 to 50% by weight, 30 to 45% by weight, 35 to 45% by weight, 40 to 45% by weight, 30 to 40% by weight, or 35 to 40% by weight based on the total weight of the oral formulation.

In addition, the API may be comprised in an amount of, for example, 50 to 500 mg, 50 to 400 mg, 50 to 300 mg, 50 to 200 mg, 50 to 100 mg, 100 to 500 mg, 100 to 400 mg, 100 to 300 mg, 100 to 200 mg, 200 to 500 mg, 200 to 400 mg, 200 to 300 mg, 300 to 500 mg, or 300 to 400 mg per unit dosage form.

The API may be comprised in an amount of, for example, 50 mg, 100 mg, 150 mg, 200 mg, 300 mg, 400 mg, or 455 mg per unit dosage form.

The API assay in the formulation of the present invention may be 95% to 105%, 96% to 105%, 97% to 105%, 98% to 105%, 99% to 105%, 100% to 105%, 95% to 100%, 96% to 100%, 97% to 100%, 97% to 100%, or 99% to 100%. In addition, the API assay may be 95% or more, 96% or more, 97% or more, 98% or more, or 99% or more, and may be 105% or less, 104% or less, 103% or less, 102% or less, 101% or less, or 100% or less.

The “API assay” refers to the content of pure API excluding impurities among API content comprised per unit dosage form. Specifically, it may have the similar meaning as the purity of the API.

The present invention provides a method for preparing an oral formulation comprising 1-(3-cyano-1-isopropyl-indol-5-yl)pyrazol-4-carboxylic acid or a pharmaceutically acceptable salt thereof, which comprises the steps of i) mixing an API selected from 1-(3-cyano-1-isopropyl-indol-5-yl)pyrazol-4-carboxylic acid or a pharmaceutically acceptable salt thereof, and a glidant to preparing a first mixture; ii) mixing pharmaceutically acceptable excipients to preparing a second mixture; and iii) formulating said first mixture and said second mixture into an oral formulation.

The steps of preparing each of the first mixture and the second mixture in the preparation method of the present invention includes a step of milling after mixing.

The glidant in the first mixture in the manufacturing method of the present invention includes SiO₂ selected from the group consisting of colloidal silicon dioxide, hydrated silicon dioxide and combinations thereof.

The pharmaceutically acceptable excipients in the second mixture in the preparation method of the present invention optionally include the aforementioned excipients.

The API assay and assay of the formulation prepared by the preparation method of the present invention are the same as the aforementioned API assay and assay of the oral formulation of the present invention, respectively.

The oral formulation may be formulated according to any tablet preparation method or granule preparation method known in the art.

In addition, the present invention provides a method for treating or preventing human xanthine oxidase-related diseases by administration of an oral formulation comprising the 1-(3-cyano-1-isopropyl-indol-5-yl)pyrazol-4-carboxylic acid or a pharmaceutically acceptable salt thereof as an API and a glidant.

As used in the present invention, the term “human xanthine oxidase-related diseases” refers to diseases that can be treated or prevented by inhibiting human xanthine oxidase, and may be, for example, hyperuricacidemia, gout, heart failure, cardiovascular diseases, high blood pressure, diabetes, diabetes-related complications, kidney diseases, inflammation, joint diseases, inflammatory bowel disease, and the like, but is not limited thereto. Examples of the diabetes-related complications may be hyperlipidemia, arteriosclerosis, obesity, high blood pressure, retinopathy, renal failure, and the like.

The term “treatment” means stopping or delaying the progression of a disease when used for a subject showing symptoms of the disease, and the term “prevention” means stopping or delaying the symptoms of the disease when administered to a subject who does not show symptoms of the disease but is at a high risk of developing the disease.

Unless otherwise indicated, it is to be understood that all numbers used in the specification and claims, whether recited or not, may be modified in all instances by the term “about.” It is also to be understood that the precise numbers used in the specification and claims form further embodiments of the present disclosure. Efforts have been made to ensure the accuracy of the numerical values disclosed in Examples. However, all measured values may inherently contain certain error values generated from the standard deviations measured by their respective measurement techniques.

### EXAMPLE AND EXPERIMENTAL EXAMPLE

Various evaluations in Examples and Comparative Examples were performed as follows.

### [Flowability Analysis]

The bulk density and tapped density of the mixture (powder) for preparing the uncoated tablets of Examples and Comparative Examples were measured. The bulk density was the volume when about 50 g of granular powder was put into the measuring cylinder, and the tapped density was measured when there was no further change in volume after lightly tapping the measuring cylinder on the floor at a constant height 100 times. The bulk density refers to the volume occupied by a certain mass of powder, and means the sum of the volume of powder and the volume of voids between particles as a total volume.

The flowability of the mixture was calculated as Carr's index in Equation (1) using the bulk density and tapped density measured above according to the method of Jinapong et al (2008).

$$CI = \frac{{ρ}_{tspped} - {ρ}_{bulk}}{{ρ}_{tapped}} \times 100. \qquad Equation ( 1 )$$

Carr's index is a measure of the compressibility of a powder and is defined as a percentage of (tapped density-bulk density)/tapped density. A higher index means that the powder is more compact and has poor flowability.

The table below shows the classification of powder flowability according to Carr's index.

TABLE 1

| Carr's index(flowability) % | Flowability |
|---|---|
| <10 | Excellent |
| 11~15 | Good |
| 16~20 | Fair |
| 21~25 | Passable |
| 26~31 | Poor |
| 32~38 | Very poor |
| >38 | Very, very poor |

### [Hardness and Friability Analysis]

The hardness and friability are used as indices that can predict the wear and tear of tablets in the handling and coating process of uncoated tablets after tableting.

For the friability test, tablets in an amount close to about 5 g are taken, the powder adhering to the tablets is removed, the mass is precisely measured, and then the tablets are placed in the drum of the friability tester. After rotating the drum 100 times, the tablets are taken out, and the powder adhering to the tablets is removed as in the beginning, and the mass is precisely measured.

Friability (%)=(mass before test−mass after test)/(mass before test)×100

In the case of hardness test, one tablet is put into a table hardness tester to measure hardness, and in this way, 10 tablets are repeated to measure the average hardness.

### [Disintegration Time Analysis]

The disintegration time is an index that affects dissolution and absorption in the body after ingestion of the formulation.

After the disintegration tester was sufficiently filled with purified water, it is adjusted to 37±2° C. Four tablets of the sample are put into the square glass pipe of the disintegration tester, it is operated in the prescribed way, and the time at which the tablet completely disintegrates and disappears is measured to obtain an average value.

### [Assay Analysis]

An assay test was conducted to evaluate the assay of the formulated formulation. The assay is an important quality characteristic of the final product, and the API average assay of the uncoated tablet was measured.

### <Analysis Condition>

Preparation of mobile phase: acetonitrile (500 m [symbol] )+purified water (500 m [symbol] )+TFA (1 m [symbol] )

Preparation of diluent: methanol (900 m [symbol] )+purified water (100 m [symbol] )

Preparation of standard solutions and test solutions: After completely dissolving the standards and samples in the diluent, they are analyzed according to the UPLC analysis method below.
Column: Waters CSH C18 (2.1 mm I.D.×100 mm L, Particle size 1.7 μm
Column temperature: 40° C.
Mobile phase: acetonitrile/H2O/TFA=500/500/1(v/v/v)
Flow rate: 0.35 m [symbol] /min
Detection: 258 nm uv
Sample amount: 1 μ [symbol]
Analysis time: 6 min

### [Dissolution Rate Analysis]

According to the dissolution test method of the 10th revision of the Korean Pharmacopoeia, the uncoated tablets of Examples and Comparative Examples below were tested for dissolution. The dissolution method was a paddle method, the stirring speed was 50 rpm, and the dissolution temperature was 37±0.5° C. The eluate was 900 ml of pH 6.8 phosphate buffer.

For the analysis conditions, the solution obtained in the above dissolution test was filtered through a 0.45 μm membrane filter and the UPLC method was used, and the concentration of 1-(3-cyano-1-isopropyl-indol-5-yl)pyrazol-4-carboxylic acid, which is an API, was analyzed.

### <Analysis Condition>

The analysis conditions are the same as the assay analysis method.

### Examples 1 to 8 and Comparative Example 1

The formulations of Examples 1 to 8 and Comparative Example 1 were prepared as uncoated tablets using the ingredients shown in Tables 2 to 4 below in the corresponding content.

Specifically, 1-(3-cyano-1-isopropyl-indol-5-yl)pyrazol-4-carboxylic acid (API) and glidant are mixed and milled to prepare a first mixture. A diluent, a disintegrant, and a lubricant, which are components not included in the first mixture, are mixed and milled to prepare a second mixture.

In the case of Examples 1 to 8 comprising the glidant, after mixing the first mixture and the second mixture, the mixture is tableted using a rotary tablet machine (Modul P, GEA, Belgium) under the conditions of pre-pressure: 5.0 kN and main pressure: 14-15 kN to obtain uncoated tablets. In the case of Comparative Example 1 that does not comprise the glidant, the process of preparing mixture 1 is omitted, and 1-(3-cyano-1-isopropyl-indol-5-yl)pyrazol-4-carboxylic acid (API) and all excipients were mixed and milled, followed by tableting in the same way as Example 1, to prepare uncoated tablets.

(If colloidal silicon dioxide is used as a glidant, it is indicated as “SiO₂.” MCC 102 used as a diluent is a microcrystalline cellulose component. PRUV® used as a lubricant is a trade name and is a sodium stearyl fumarate component).

TABLE 2

| | | Example 1 | | Example 2 | | Example 3 | | Example 4 | |
|---|---|---|---|---|---|---|---|---|---|
| | | Content | Content | Content | Content | Content | Content | Content | Content |
| Item | Ingredient | mg/T | ratio % | mg/T | ratio % | mg/T | ratio % | mg/T | ratio % |
| API | | 100.0 | 45.5 | 100.0 | 45.5 | 100.0 | 45.5 | 100.0 | 45.5 |
| Diluent | MCC 102 | 105.4 | 47.9 | 104.8 | 47.6 | 104.1 | 47.3 | 103.7 | 47.1 |
| | (microcrystalline | | | | | | | | |
| | cellulose) | | | | | | | | |
| Disintegrant | Crospovidone | 9.7 | 4.4 | 9.7 | 4.4 | 9.7 | 4.4 | 9.7 | 4.4 |
| Glidant | Colloidal | 0.5 | 0.2 | 1.1 | 0.5 | 1.8 | 0.8 | 2.2 | 1.0 |
| | silicon | | | | | | | | |
| | dioxide | | | | | | | | |
| | (SiO₂) | | | | | | | | |
| Lubricant | PRUV ® | 4.4 | 2.0 | 4.4 | 2.0 | 4.4 | 2.0 | 4.4 | 2.0 |

| | | | | | | | | |
|---|---|---|---|---|---|---|---|---|
| Total weight of tablets | 220.0 | 100.0 | 220.0 | 100.0 | 220.0 | 100.0 | 220.0 | 100.0 |
| (mg) | | | | | | | | |

TABLE 3

| | | Example 5 | | Example 6 | | Example 7 | | Example 8 | |
|---|---|---|---|---|---|---|---|---|---|
| | | Content | Content | Content | Content | Content | Content | Content | Content |
| Function | Type | mg/T | ratio % | mg/T | ratio % | mg/T | ratio % | mg/T | ratio % |
| API | | 100.0 | 45.5 | 100.0 | 50.0 | 100.0 | 54.9 | 100.0 | 59.9 |
| Diluent | MCC 102 | 104.8 | 47.6 | 84.8 | 42.4 | 66.8 | 36.7 | 51.8 | 31.0 |
| | (microcrystalline | | | | | | | | |
| | cellulose) | | | | | | | | |
| Disintegrant | Crospovidone | 9.7 | 4.4 | 9.7 | 4.9 | 9.7 | 5.3 | 9.7 | 5.8 |
| Glidant | Colloidal | — | — | 1.1 | 0.6 | 1.1 | 0.6 | 1.1 | 0.7 |
| | silicon | | | | | | | | |
| | dioxide(SiO₂) | | | | | | | | |
| | Talc | 1.1 | 0.5 | — | — | — | — | — | — |
| Lubricant | PRUV ® | 4.4 | 2.0 | 4.4 | 2.2 | 4.4 | 2.4 | 4.4 | 2.6 |

| | | | | | | | | |
|---|---|---|---|---|---|---|---|---|
| Total weight of | 220.0 | 100.0 | 200.0 | 100.0 | 182.0 | 100.0 | 167.0 | 100.0 |
| tablets(mg) | | | | | | | | |

TABLE 4

Comparative Example 1

| | | | Content | Content ratio |
|---|---|---|---|---|
| | Function | Type | mg/T | % |
| API | | | 100.0 | 45.5 |
| | Diluent | MCC 102 | 105.9 | 48.1 |
| | | (microcrystalline | | |
| | | cellulose) | | |
| | Disintegrant | Crospovidone | 9.7 | 4.4 |
| | Lubricant | PRUV ® | 4.4 | 2.0 |

| | | |
|---|---|---|
| Total weight of tablets (mg) | 220.0 | 100.0 |

### Experimental Example 1: Characteristic Analysis of Formulation Depending on Glidant Content

The physical properties of the oral formulation comprising 1-(3-cyano-1-isopropyl-indol-5-yl)pyrazol-4-carboxylic acid as an API, depending on the inclusion and content of a glidant of SiO₂ as an excipient were analyzed by the aforementioned analysis method.

Specifically, the physical properties of the mixture state before formulation (bulk density, tapped density and Carr's index) and the physical properties of the uncoated tablet state after tableting the mixture (hardness, brittleness, disintegration time, assay and dissolution rate) were analyzed. The results are summarized in Table 5 below.

The bulk density, tapped density, and Carr's index did not show any significant difference depending on the inclusion and content of the glidant (SiO₂) in the mixture state before the uncoated tablet was prepared.

It has been known that when glidant is added to the conventional pharmaceutical formulation, the flowability is improved. However, in the case of the API (1-(3-cyano-1-isopropyl-indol-5-yl)pyrazol-4-carboxylic acid) of the present invention, the flowability was not significantly increased even when the glidant was comprised.

However, as a result of measuring the hardness and friability of the uncoated tablet, when SiO₂ is comprised (Examples 1 to 4) compared to the case without SiO₂ (Comparative Example 1), the hardness is 6.7 kP or higher, which satisfies 6.0 kP which is a target hardness of the uncoated tablet. On the other hand, in the case of Comparative Example 1, it was only 5.4 kP, which did not reach the target hardness of the uncoated tablet, making it unsuitable for formulation into tablets (Table 5).

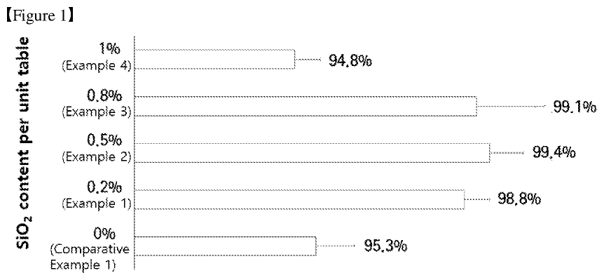

In addition, as a result of measuring the API assay of the uncoated tablet, as shown in Table 6 and FIG. 1 below, in the case of Comparative Example 1, the average assay is 95.3%, and when comprising 0.2 to 0.8% by weight of SiO₂, it is increased up to 98.8 to 99.4% (Examples 1 to 3). However, in the case of Example 4 comprising 1% by weight of SiO₂, it is reduced to 94.8% similar to Comparative Example 1, indicating that the preferred content range of SiO₂ is 0.2 to 0.8% by weight based on the total weight of the formulation.

Next, in the case of dissolution rate, Comparative Example 1 has a low hardness, so it shows early dissolution, but shows a constant dissolution rate thereafter. However, when SiO₂ was comprised, the slope showed a constant increase, and in particular, Example 2 (0.5% by weight of SiO₂) showed the highest tendency for the 60-minute dissolution rate (Table 5).

TABLE 5

| | | | | | | Com- |
|---|---|---|---|---|---|---|
| | | Exam- | Exam- | Exam- | Exam- | parative |
| | | ple | ple | ple | ple | Exam- |
| Item | | 1 | 2 | 3 | 4 | ple 1 |
| SiO₂ content (%) (based | | 0.2 | 0.5 | 0.8 | 1 | 0 |
| on total tablet) | | | | | | |
| Bulk density (g/ml) | | 0.44 | 0.39 | 0.41 | 0.41 | 0.4 |
| Carr's index (%) | | 23.0 | 25.0 | 24.0 | 24.0 | 25.0 |
| Hardness (kP) | | 6.7 | 7.3 | 7.5 | 7.2 | 5.4 |
| Friability (%) | | 0.15 | 0.05 | 0.04 | 0.03 | 0.05 |
| Disintegration time (sec) | | 12 | 10 | 10 | 12 | 10 |
| Assay | Average | 98.8 | 99.4 | 99.1 | 94.8 | 95.3 |
| | assay (%) | | | | | |
| Dissolution | Average | 68.3 | 71.8 | 66.1 | 67.9 | 84.0 |
| rate | dissolution | | | | | |
| | rate (15 min) | | | | | |
| | (%) | | | | | |
| | Average | 82.9 | 88.6 | 82.2 | 80.9 | 88.5 |
| | dissolution | | | | | |
| | rate (30 min) | | | | | |
| | (%) | | | | | |
| | Average | 86.9 | 95.8 | 87.8 | 85.9 | 88.8 |
| | dissolution | | | | | |
| | rate (60 min) | | | | | |
| | (%) | | | | | |

### Experimental Example 2: Characteristic Analysis of Formulation Depending on Type of Glidant

In order to analyze the characteristics depending on the types of glidant, the physical properties of example 2 and example 5 were analyzed. Example 2 shows best physical properties in table 5, and Example 5 was prepared by changing SiO₂ to talc while maintaining the glidant content (0.5% by weight) from Example 2.

As shown in Table 6 below, in the case of Example 5 comprising talc as a glidant in the mixture state before the uncoated tablet was prepared, the bulk density showed no difference compared with Example 2 and Comparative Example 1, but Carr's index was calculated to be 28%. Therefore, in the case of Example 5, Carr's index is “poor” category and is not suitable for direct pressing. In addition, in the case of Example 5, the hardness of the uncoated tablet is 5.1 kP, which does not reach the target hardness of 6.0 kP, and the friability is also three times higher than in Example 2, and thus it has physical properties that are not suitable for the tablet.

It can be seen that the average assay of Example 5 is also lowered compared to Example 2.

Therefore, it can be seen that when SiO₂ is used as a glidant, the overall physical properties are improved compared to talc.

TABLE 6

| Item | | Example 2 | Example 5 |
|---|---|---|---|
| Glidant content (%) (based on total tablet) | | 0.5 | 0.5 |
| Bulk density (g/ml) | | 0.39 | 0.41 |
| Carr's index (%) | | 25.0 | 28.0 |
| Hardness (kP) | | 7.3 | 5.1 |
| Friability (%) | | 0.05 | 0.14 |
| Disintegration time (sec) | | 10 | 8 |
| Assay | Average assay (%) | 99.4 | 96.5 |

### Experimental Example 3: Characteristic Analysis Depending on API Content

In order to prepare an uncoated tablet comprising an API of 50% by weight or more, the characteristics depending on the increase of API content (Examples 6 to 8) based on the content (45.5% by weight) of Example 2 were analyzed.

According to Table 7, since the density of the API is increased compared to the diluent as the API content is increased, the bulk density tends to slightly increase as the API content is increased, but the flowability itself does not show a significant difference.

However, as the API content is increased, the hardness of the uncoated tablet is weakened and friability is increased due to the decrease in tableting properties (reduction in compressibility of the tablet), and in particular, Example 8 (API content: 60% by weight) showed a hardness of 3.0 kP and a friability of 0.2% or more, and thus it was not suitable for formulating into tablets (Table 7).

Also, as a result of measuring the API assay of the uncoated tablet, Examples 2, 6, and 7 showed an average assay of 99% or more, whereas Example 8 showed an average assay of 94.6% (Table 7).

Taken together, when the API content is 55% by weight or less, the average assay is 99% or more, but when the API content is 60% by weight, the average assay is significantly lowered to 94.6%, and when the API content is more than 50%, even if the content is excellent, the hardness of the uncoated tablet is lowered and the friability is increased. Therefore, it can be seen that when the content of the API is 55% by weight or more, it is not desirable to formulate it as a tablet.

TABLE 7

| | | Example | Example | Example | Example |
|---|---|---|---|---|---|
| Item | | 2 | 6 | 7 | 8 |
| API content (%) (based | | 45.5 | 50 | 55 | 60 |
| on total tablet) | | | | | |
| Bulk density (g/ml) | | 0.39 | 0.43 | 0.43 | 0.44 |
| Carr's index (%) | | 25.0 | 25.0 | 24.0 | 23.0 |
| Hardness (kP) | | 7.3 | 5.4 | 4.0 | 3.0 |
| Friability (%) | | 0.05 | 0.08 | 0.10 | 0.22 |
| Disintegration time (sec) | | 10 | 10 | 10 | 7 |
| Assay | Average | 99.4 | 99.6 | 99.3 | 94.6 |
| | assay (%) | | | | |

Up to now, preferred examples of the present invention have been mainly looked at. It will be understood by those of ordinary skill in the art to which the present invention pertains that the present invention can be implemented in a modified form without departing from the essential characteristics of the present invention. Therefore, the examples disclosed above are to be considered in an illustrative rather than a restrictive sense. The scope of the present invention is indicated in the claims rather than the foregoing description, and all differences within the scope equivalent thereto should be construed as being included in the present invention.

1. An oral formulation comprising an active pharmaceutical ingredient (API) selected from 1-(3-cyano-1-isopropyl-indol-5-yl)pyrazol-4-carboxylic acid or a pharmaceutically acceptable salt thereof; and a glidant.

2. The oral formulation according to claim 1, wherein the glidant is selected from the group consisting of colloidal silicon dioxide, hydrated silicon dioxide and combinations thereof.

3. The oral formulation according to claim 2, wherein the content of the glidant is greater than 0% by weight and less than 1% by weight based on the total weight of the formulation.

4. The oral formulation according to claim 3, wherein the content of the glidant is 0.2 to 0.8% by weight based on the total weight of the formulation.

5. The oral formulation according to claim 1, wherein the oral formulation further comprises one or more excipients selected from a diluent, a disintegrant, a binder and a lubricant.

6. The oral formulation according to claim 1, wherein the content of the API is 30 to 55% by weight based on the total weight of the formulation.

7. The oral formulation according to claim 6, wherein the content of the API is 40 to 50% by weight based on the total weight of the formulation.

8. The oral formulation according to claim 1, wherein the content of the API is 50 mg, 100 mg, 200 mg, or 300 mg per unit dosage form.

9. The oral formulation according to claim 1, wherein the assay of the API is 97% or more.

10. A method for preparing an oral formulation comprising 1-(3-cyano-1-isopropyl-indol-5-yl)pyrazol-4-carboxylic acid or a pharmaceutically acceptable salt thereof, which comprises the steps of
i) mixing 1-(3-cyano-1-isopropyl-indol-5-yl)pyrazol-4-carboxylic acid or a pharmaceutically acceptable salt thereof as an API; and a glidant to preparing a first mixture;
ii) mixing pharmaceutically acceptable excipients to preparing a second mixture; and
iii) formulating said first mixture and said second mixture into an oral formulation.

11. The method for preparing an oral formulation according to claim 10, wherein the content of the glidant is 0.2 to 0.8% by weight based on the total weight of the formulation.

12. The method for preparing an oral formulation according to claim 10, wherein the content of the API is 40 to 50% by weight based on the total weight of the formulation.

13. An oral formulation prepared by the method of claim 10, wherein the assay of the above API is 97% or more.
